(19) United States
(12) Patent Application Publication
(10) Pub. No.: US 2025/0346940 A1
(43) Pub. Date: Nov. 13, 2025

(54) DIELECTROPHORESIS DETECTION DEVICE FOR DETECTION AND QUANTIFICATION OF BACTERIA IN WATER

(71) Applicant: ZHEJIANG NORMAL UNIVERSITY, Jinhua (CN)

(72) Inventors: CLARENCE AUGUSTINE TECK HUO TEE, Jinhua (CN); BURHANUDDIN YEOP MAJLIS, Selangor (MY); MUHAMAD RAMDZAN BUYONG, Selangor (MY); WEY PING YEO, Selangor (MY)

(21) Appl. No.: 19/020,818

(22) Filed: Jan. 14, 2025

(30) Foreign Application Priority Data
May 8, 2024 (CN) 202410560997.X

(51) Int. Cl. C12Q 1/06; G01N 27/447

(52) U.S. Cl. CPC C12Q 1/06; G01N 27/44791

(57) ABSTRACT

Disclosed is a dielectrophoresis detection device for detection and quantification of bacteria in water. The device includes a jig, the jig includes a top chip layer, a base chip layer arranged opposite to the top chip layer, an accommodating part is arranged between the top chip layer and the base chip layer, a semiconducting microelectrode chip is arranged in the accommodating part, a gasket layer that fits the semiconducting microelectrode chip is further arranged in the accommodating part, and the semiconducting microelectrode chip is provided with a dielectrophoretic actuator microelectrode part and an impedance sensor microelectrode part arranged next to the dielectrophoretic actuator microelectrode part. The present disclosure, through quantitative integration of dielectricity and impedance, is capable of detecting highly virulent and antibiotic-resistant bacterial pathogens.

### CROSS-REFERENCE TO RELATED APPLICATIONS

This application claims priority to Chinese Patent Application No. 202410560997.X, filed on May 8, 2024, which is hereby incorporated by reference in its entirety.

### TECHNICAL FIELD

The present disclosure relates to the technical field of detection devices, and in particular to the technical field of dielectrophoresis detection devices for detection and quantification of bacteria in water.

### BACKGROUND

Widespread water pollution is jeopardizing our health. Deaths caused by unsafe water are more than total deaths from wars and all other forms of violence each year. Meanwhile, our drinkable water sources are limited. Only less than 1% of freshwater on the Earth is accessible to us. Without action, the world will encounter more severe challenges by 2050, and global demand for freshwater is expected to increase by one third compared to now.

Microfluidics is not only a science of studying a behavior of fluids passing through microchannels, but also a technology of manufacturing microminiaturized devices containing chambers and tunnels through which fluids flow or are confined. A volume of fluid processed by microfluidics is extremely small, down to femtoliters (fL), which is a quadrillionth of a liter. A microfluidic chip is a pattern of microchannels molded or engraved. A network of microchannels incorporated into the microfluidic chip is linked to a macro environment through a plurality of holes of different dimensions hollowed out through the chip. Fluids are injected into and discharged from the microfluidic chip just through these pathways.

Fluids are directed, mixed, separated or manipulated to obtain a multiplexed, automated and high-throughput system.

The design of the microchannel network must be precisely elaborated to achieve desired features (such as lab-on-a-chip, pathogen detection, electrophoresis, DNA analysis and the like).

Dielectrophoresis (DEP) is a phenomenon in which a force is exerted on dielectric particles under the action of a non-uniform electric field. This force does not require particle charging. In the presence of an electric field, all particles exhibit dielectrophoretic activity. This mechanism can be used to detect bacteria.

Over the past four decades, DEP technology has been explored for research purposes to discover extensions and limitations in use of technical kinetic methods for cell identification. DEP is an important subject that has been innovative due to its potential in manipulating microparticles, nanoparticles, and cell biology. DEP continuous flow microfluidics can be used to identify and quantify microorganisms including bacteria, viruses, and protozoa through dielectric electrochemical properties (DEP) techniques. Based on the continuous flow microfluidics, the device can be applied to the fields of biosensor testing, cell culture and analysis, dielectric testing, impedance detection, and interfacing with silicon chips and sensors.

A cellular impedance is calculated based on a difference between a baseline voltage and a voltage measured after a battery is connected to an electrode, which eliminates a contribution of an electrode-electrolyte combination and parasitic elements. An impedance mechanism can be used to calculate a concentration of particles in a culture medium or sample.

Both elements are suitable for detecting and quantifying quantum amounts of six highly virulent and antibiotic-resistant bacterial pathogens including: Enterococcus faecalis, Staphylococcus aureus, Klebsiella pneumoniae, Acinetobacter baumannii, Pseudomonas aeruginosa and Enterobacter spp.

The Malaysian patent PI 2020006334 is a prior art of DEP technology. The patent discloses an on-chip DEP device for rapid and high-sensitivity detection of diseases caused by or associated with viral infection in a subject, including influenza A virus and mutations thereof, and coronaviruses causing severe acute respiratory syndrome (SARS), including MERS-COV, SARS-COV-1 and SARS-COV-2 or COVID-19. The on-chip DEP device includes an electrode array and a microfluidic microchannel, where the electrode array includes tapered DEP microelectrodes in pairs that are arranged at a regular interval along a fluid flow path through which a sample fluid flows. The tapered DEP microelectrode is formed as a frustum structure with a tapered profile angle of 5°-90° and high-intensity electric field spots, which facilitate selective detection and rapid manipulation of particles on a y-axis and a z-axis in the sample fluid, where the particles are affected by an x-axis. A DEP force is used to achieve manipulation and separation of particles between a planar surface of the frustum structure and the tapered DEP microelectrodes. The sample fluid is saliva collected from the subject.

However, in the above previous ARTs, quantitative integration of dielectric actuators and impedance sensors is not achieved. Therefore, rapid and selective detection of ESKAPE pathogens cannot be achieved. Therefore, an invention is needed to overcome the defects of existing arrangements of the prior art.

### SUMMARY

In order to solve the problems in the prior art, an objective of the present disclosure is to provide a dielectrophoresis detection device for detection and quantification of bacteria in water, and the device, through quantitative integration of dielectricity and impedance, is capable of detecting highly virulent and antibiotic-resistant bacterial pathogens.

In order to achieve the above objective, the present disclosure provides a dielectrophoresis detection device for detection and quantification of bacteria in water, and the device includes a jig, where the jig includes a top chip layer, a base chip layer arranged opposite to the top chip layer, an accommodating part arranged between the top chip layer and the base chip layer, a semiconducting microelectrode chip arranged in the accommodating part, and a gasket layer that fits the semiconducting microelectrode chip further arranged in the accommodating part, where the semiconducting microelectrode chip is provided with a dielectrophoretic actuator microelectrode part and an impedance sensor microelectrode part arranged next to the dielectrophoretic actuator microelectrode part, the dielectrophoretic actuator microelectrode part is composed of two rows of dielectrophoresis microelectrodes arranged opposite to each other, a dielectrophoresis channel for fluid to pass through is arranged between the two rows of dielectrophoresis microelectrodes, one end of the dielectrophoretic actuator microelectrode part is provided with a dielectrophoresis inlet communicated with the dielectrophoresis channel, and the other end thereof is provided with a dielectrophoresis outlet communicated with the dielectrophoresis channel;

the impedance sensor microelectrode part is composed of two rows of impedance microelectrodes arranged opposite to each other, an impedance channel for fluid to pass through is arranged between the two rows of impedance microelectrodes, one end of the impedance sensor microelectrode part is provided with an impedance inlet communicated with the impedance channel, the other end thereof is provided with an impedance outlet communicated with the impedance channel, and the impedance inlet is communicated with the dielectrophoresis outlet; and

a pressure pump for pumping fluid into the dielectrophoresis inlet is arranged at the dielectrophoresis inlet, and an extraction pump for extracting fluid from the impedance outlet is arranged at the impedance outlet.

Preferably, the number of the dielectrophoresis outlets is three, i.e., a first outlet, a second outlet, and a third outlet are included respectively.

Preferably, a liquid reservoir is further arranged on the semiconducting microelectrode chip close to the dielectrophoresis outlet.

Preferably, the impedance inlet is arranged at one end of the semiconducting microelectrode chip close to the dielectrophoresis outlet.

Preferably, the dielectrophoretic actuator microelectrode part is configured for detecting, manipulating, and classifying bacteria.

Preferably, the impedance sensor microelectrode part is configured for quantifying bacteria classified through the dielectrophoretic actuator microelectrode part.

The dielectrophoresis detection device for detection and quantification of bacteria in water of the present disclosure has the following beneficial effects: The present disclosure achieves rapid, in-situ and sensitive applications, and is capable of detecting highly virulent and antibiotic-resistant bacterial pathogens, including Enterococcus faecium, Staphylococcus aureus, Klebsiella pneumoniae, Acinetobacter baumannii, Pseudomonas aeruginosa, and Enterobacter spp. By using microliter amounts of samples and preserving the samples, the user can perform in situ detection of bacterial pathogens rapidly and at low cost with minimal laboratory equipment. The present disclosure provides a more efficient detection solution, and the solution is easily applied and validated in practical applications. The solution can become a solution for stakeholders such as communities and food and beverage manufacturers as users of clean water, wastewater treatment enterprises producing clean water, and governments providing high-quality water to communities. Based on different DEP responses, bacterial cells can be manipulated according to the interest of an experimental target, and impedance-based concentration measurement can be performed for quantification. The present disclosure provides an alternative solution for bacteriological water analysis by targeting ESKAPE pathogens and potential antimicrobial resistance bacteria. The present disclosure can also potentially be used for antimicrobial resistance bacteria, because through previous work, researchers can characterize drug-resistant and sensitive bacteria by dielectrophoresis. Use of antibiotics can change the characteristics of bacteria, particularly cell wall characteristics of drug-resistant bacteria and sensitive bacteria, such that the DEP responses can be changed based on changes in the bacteria. The present disclosure has unlimited potential and can be customized for the experimental target.

The features and advantages of the present disclosure will be described in detail with reference to the examples and the accompanying drawings.

### BRIEF DESCRIPTION OF DRAWINGS

FIG. 1 is a schematic diagram of a complete structure of a dielectrophoresis detection device for detection and quantification of bacteria in water according to the present disclosure.

FIG. 2 is a schematic diagram of a top view of a semiconducting microelectrode chip of a dielectrophoresis detection device for detection and quantification of bacteria in water according to the present disclosure.

Reference numerals in the figures:

100-jig; 101-top chip layer; 103-base chip layer; 105-gasket layer; 200-semiconducting microelectrode chip; 201-dielectrophoretic actuator microelectrode part; 201-a dielectrophoresis inlet; 201-b first outlet; 201-e pressure pump; 201-c second outlet; 201-d third outlet; 203-b impedance outlet; and 204-liquid reservoir.

### DESCRIPTION OF EMBODIMENTS

To make the objectives, technical solutions and advantages of the present disclosure clearer, the present disclosure will be described in further detail below with reference to the accompanying drawings and the examples. It should be understood that the specific examples described herein are merely illustrative of the present disclosure and are not intended to limit the present disclosure. In addition, in the following descriptions, descriptions of well-known structures and technologies are omitted in order to avoid unnecessarily obscuring the concepts of the present disclosure.

In the descriptions of the present disclosure, it should be noted that when an element is referred to as being “fixed to” or “arranged on” another element, the element may be directly or indirectly on another element. When an element is referred to as being “connected to” another element, the element may be directly or indirectly connected to another element.

In the descriptions of the present disclosure, it should be noted that the terms “center”, “length”, “width”, “thickness”, “upper”, “lower”, “front”, “back”, “left”, “right”, “vertical”, “horizontal”, “top”, “bottom”, “inner”, “outer”, etc. indicate orientation or position relations based on those shown in the accompanying drawings, or of common placement when the product of the present disclosure is used, which are only for ease of description of the present disclosure and for simplicity of description, and are not intended to indicate or imply that the referenced device or element must have a particular orientation and be constructed and operated in a particular orientation, and thus may not be construed as a limitation on the present disclosure. Moreover, the terms “first”, “second”, “third”, etc. are used merely to distinguish between descriptions and may not be construed as indication or implication of relative importance. Thus, a feature defined with “first” and “second” may explicitly or implicitly include one or more of the features. In the description of the present disclosure, “a plurality of” means two or more, unless expressly specified otherwise. “Several” means one or more, unless expressly specified otherwise.

In the description of the present disclosure, it should be further noted that, unless otherwise clearly specified, meanings of terms “arrange”, “mount”, “connected” and “connect” should be understood in a board sense. For example, the connection may be a fixed connection, a detachable connection, an integral connection; may be a mechanical connection or an electrical connection; may be a direct connection or an indirect connection by using an intermediate medium; or may be intercommunication between two components. For those of ordinary skill in the art, the specific meanings of the above terms in the present disclosure may be understood according to specific circumstances.

### Example 1

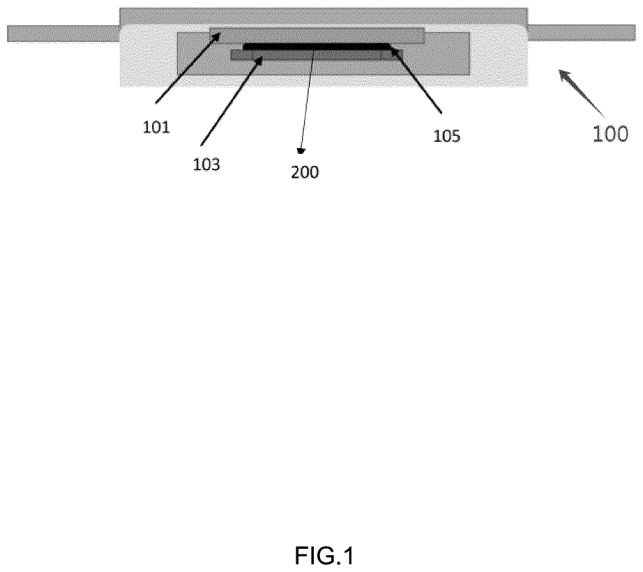

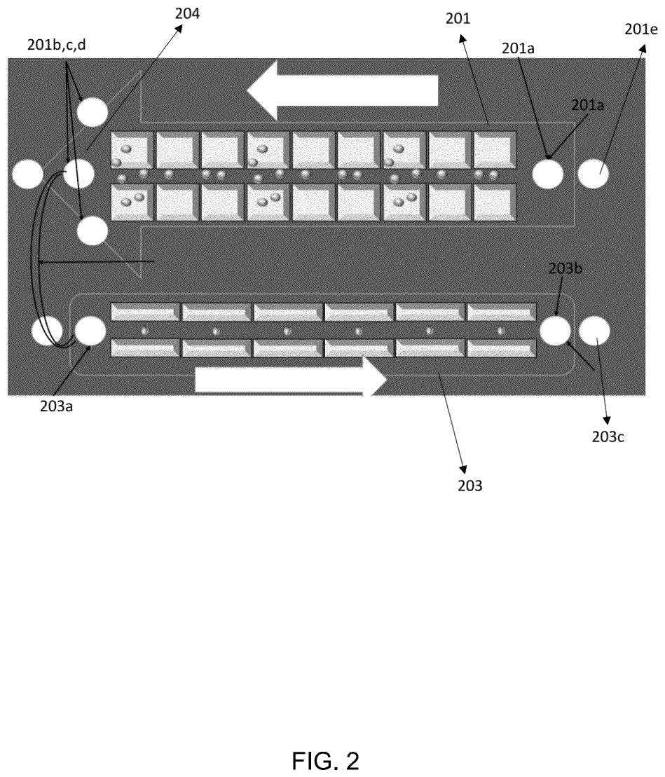

With reference to FIGS. 1 and 2, the present disclosure provides a dielectrophoresis detection device for detection and quantification of bacteria in water. The present disclosure is usually dielectrically ball-contacted with water bacteria disease, provided with a dielectrophoretic actuator with a resistance sensor, and used for quantitative equipment and continuous flow fluidity detection. The device includes a jig 100, the jig 100 includes a top chip layer 101, a base chip layer 103 arranged opposite to the top chip layer 101, an accommodating part is arranged between the top chip layer 101 and the base chip layer 103, a semiconducting microelectrode chip 200 is arranged in the accommodating part, a gasket layer 105 that fits the semiconducting microelectrode chip 200 is further arranged in the accommodating part, the semiconducting microelectrode chip 200 is provided with a dielectrophoretic actuator microelectrode part 201 and an impedance sensor microelectrode part 203 arranged next to the dielectrophoretic actuator microelectrode part 201, the dielectrophoretic actuator microelectrode part 201 is composed of two rows of dielectrophoresis microelectrodes arranged opposite to each other, a dielectrophoresis channel for fluid to pass through is arranged between the two rows of dielectrophoresis microelectrodes, one end of the dielectrophoretic actuator microelectrode part 201 is provided with a dielectrophoresis inlet 201a communicated with the dielectrophoresis channel, the other end thereof is provided with a dielectrophoresis outlet communicated with the dielectrophoresis channel, the impedance sensor microelectrode part 203 is composed of two rows of impedance microelectrodes arranged opposite to each other, an impedance channel for fluid to pass through is arranged between the two rows of impedance microelectrodes, one end of the impedance sensor microelectrode part 203 is provided with an impedance inlet 203a communicated with the impedance channel, the other end thereof is provided with an impedance outlet 203b communicated with the impedance channel, and the impedance inlet 203a is communicated with the dielectrophoresis outlet; and a pressure pump 201E for pumping fluid into the dielectrophoresis inlet 201a is arranged at the dielectrophoresis inlet 201a, and an extraction pump 203c for extracting fluid from the impedance outlet 203b is arranged at the impedance outlet 203b. The number of the dielectrophoresis outlets is three, i.e., a first outlet 201b, a second outlet 201c, and a third outlet 201d are included respectively, and a liquid reservoir 204 is further arranged on the semiconducting microelectrode chip 200 close to the dielectrophoresis outlet.

In a continuous flow microfluidic flow process, the dielectrophoretic actuator microelectrode part 201 is configured for detecting, manipulating, and classifying bacteria. Differences in characteristics of bacteria in sizes, shapes, cell walls, cytoplasm and membranes contribute to conductivity and permittivity and cause different polarization factors across a frequency range, where different dielectric responses are exhibited in the frequency range. Based on different DEP responses, bacterial cells can be manipulated, separated and classified according to interests of target bacteria. The pressure pump 201E pumps fluid in the dielectrophoresis inlet 201a, and fluid particles move toward the three outlets, i.e., the first outlet 201b, the second outlet 201c, and the third outlet 201d.

The impedance sensor microelectrode part 203 is configured for quantifying bacteria classified through the dielectrophoretic actuator microelectrode part 201. The impedance sensor will quantify proliferation of bacterial cells in liquid and convert it into a bacterial concentration. When a peak value of the bacterial concentration is detected, an impedance measurement value will increase. After the fluid flows out from the first outlet 201b, the second outlet 201c, and the third outlet 201d, the fluid will move inside the impedance inlet 203a and flow to the impedance outlet 203b. The extraction pump 203c is configured for extracting the fluid from the impedance outlet 203b.

Flow of the fluid from the first outlet 201b, the second outlet 201c, and the third outlet 201d to the impedance inlet 203a helps to measure a flow rate of the fluid, and the flow of the fluid from the impedance inlet 203a to the impedance outlet 203b helps to measure an impedance, which indicates a concentration of bacteria in the liquid.

In the impedance sensor microelectrode part 203, the fluid experiences two scenarios: positive DEP (pDEP) particle attraction and negative DEP (nDEP) particle repulsion from electrodes.

During pDEP, particles will be attracted to the electrodes, such that the fluid can pass through a channel without target particles.

Through nDEP, particles are repelled from the electrodes, such that the fluid can pass through a channel with the target particles.

By targeting the target particles, DEP can be performed by manipulating particles in the fluid, and other particles can be filtered out through nDEP. Then the fluid with the target particles will be pumped into the impedance sensor microelectrode part 203 for impedance sensor measurement. The fluid with other particles will be collected in the liquid reservoir 204.

The present disclosure provides an alternative to bacteriological water analysis by targeting ESKAPE pathogens. By using microliter amounts of samples and preserving the samples, the user can perform in situ detection of bacterial pathogens rapidly and at low cost with minimal laboratory equipment. The present disclosure provides a more efficient detection solution, and the solution is easily applied and validated in practical applications. The solution can become a solution for stakeholders such as communities and food and beverage manufacturers as users of clean water, wastewater treatment enterprises producing clean water, and governments providing high-quality water to communities.

Differences exist between these bacterial pathogens, such that this may play a role in differences between dielectrophoresis conductivity and permittivity. These differences may exist in various parts of cells, where differences in the conductivity and permittivity of outer layers, cell walls, cytoplasm and membranes may cause an influence of polarization factors, so as to result in different DEP responses. Based on different DEP responses, bacterial cells can be manipulated according to the interest of an experimental target, and impedance-based concentration measurement can be performed for quantification. The present disclosure provides an alternative solution for bacteriological water analysis by targeting ESKAPE pathogens and potential antimicrobial resistance bacteria.

The present disclosure can also potentially be used for antimicrobial resistance bacteria, because through previous work, researchers can characterize drug-resistant and sensitive bacteria by dielectrophoresis. Use of antibiotics can change the characteristics of bacteria, particularly cell wall characteristics of drug-resistant bacteria and sensitive bacteria, such that the DEP responses can be changed based on changes in the bacteria. The present disclosure has unlimited potential and can be customized for the experimental target.

### Example 2

This is a DEP manipulation and sorting procedure for micron and nanometer-sized biological samples. This is a closed-loop procedure of dielectrophoresis for actuator manipulation and separation, and quantification of sensor documents. The procedure applies to micron to nanometer sized particles (a biological sample range) such as cells, bacteria, proteins and viruses. Effectiveness of dielectrophoresis is claimed to proceed in four stages: (1) polarization factor modeling and simulation; (2) main experiments; (3) qualitative observation; and (4) quantitative analysis. At each stage, data is observed to be related to a closed loop, and dielectrophoresis results are validated to produce a reliable source for DEP application in micron to nanometer sized biological particles.

Stage (1) Polarization factor modeling and simulation: this stage can start from selection of DEP polarization factors with Clausius-Mossotti factors based on particles and media, or from experimental DEP based on knowledge of biological characteristics. When parameters of cell particles and media are well known, DEP polarization and trajectory factors can be completed, and the conductivity and permittivity are influenced by an input frequency.

Stage (2) Main experiments: refer to experiments for CMF-based characterizing or validating of DEP responses of pDEP, nDEP and fxo through dielectrophoresis. When parameters of the particles are unclear, DEP experiments can be performed to characterize DEP response spectra of particles of pDEP, nDEP and fxo.

Stage (3) Qualitative observation: refers to DEP analysis of images and videos on an x-axis and a y-axis, and z stacks on a z-axis. The analysis in the third stage requires a combination of the images, the videos and the z stacks.

Stage (4) Quantitative analysis: All images, videos, and z stacks will be used to analyze velocity and head-to-tail trajectories of particles.

Dielectrophoresis data will be supported and validated in each of these stages,

because resulting outputs of velocity and trajectories will be aligned with a CMF curve model according to results of polarization factor model simulation. A process of closed-loop iterations for over three times requires efficiency of a DEP application as a DEP actuator in terms of detection, manipulation, and sorting. Iterations for three to five times will enhance sensitivity and selectivity of quantitative combination of DEP as an actuator with DEP as a sensor for a biological application within a micron and nanometer range.

Standard parts referenced in the present disclosure can be purchased from the market, and are connected through specific mature conventional connection methods of the prior art for bolts, rivets, welding and the like. Inner components such as electric sliding rail carriages, cylinders, welding machines, electric telescopic rods and controllers are of conventional models in the prior art, and their internal structures belong to structures of the prior art. Workers can perform normal operations according to manuals of the prior art, and conventional circuit connection methods of the prior art are adopted, so details are not described herein.

It should be noted that the above examples have been described herein, but are not intended to limit the patent scope of the present disclosure. Therefore, changes and modifications of made to the example described herein based on the innovative concepts of the present disclosure, or any equivalent structure or equivalent process transformation made by using the description of the present disclosure and the contents of the accompanying drawings, with the above technical solution directly or indirectly used in other related technical fields, are all included in the protection scope of the present disclosure.

What is claimed is:

1. A dielectrophoresis detection device for detection and quantification of bacteria in water, comprising a jig (100), wherein the jig (100) comprises a top chip layer (101), a base chip layer (103) arranged opposite to the top chip layer (101), an accommodating part arranged between the top chip layer (101) and the base chip layer (103), a semiconducting microelectrode chip (200) arranged in the accommodating part, and a gasket layer (105) that fits the semiconducting microelectrode chip (200) further arranged in the accommodating part, wherein the semiconducting microelectrode chip (200) is provided with a dielectrophoretic actuator microelectrode part (201) and an impedance sensor microelectrode part (203) arranged next to the dielectrophoretic actuator microelectrode part (201), the dielectrophoretic actuator microelectrode part (201) is composed of two rows of dielectrophoresis microelectrodes arranged opposite to each other, a dielectrophoresis channel for fluid to pass through is arranged between the two rows of dielectrophoresis microelectrodes, one end of the dielectrophoretic actuator microelectrode part (201) is provided with a dielectrophoresis inlet (201a) communicated with the dielectrophoresis channel, and the other end thereof is provided with a dielectrophoresis outlet communicated with the dielectrophoresis channel;
the impedance sensor microelectrode part (203) is composed of two rows of impedance microelectrodes arranged opposite to each other, an impedance channel for fluid to pass through is arranged between the two rows of impedance microelectrodes, one end of the impedance sensor microelectrode part (203) is provided with an impedance inlet (203a) communicated with the impedance channel, the other end thereof is provided with an impedance outlet (203b) communicated with the impedance channel, and the impedance inlet (203a) is communicated with the dielectrophoresis outlet; and
a pressure pump (201E) for pumping fluid into the dielectrophoresis inlet (201a) is arranged at the dielectrophoresis inlet (201a), and an extraction pump (203c) for extracting fluid from the impedance outlet (203b) is arranged at the impedance outlet (203b).

2. The dielectrophoresis detection device for detection and quantification of bacteria in water according to claim 1, wherein the number of the dielectrophoresis outlets is three, i.e., a first outlet (201b), a second outlet (201c), and a third outlet (201d) are comprised respectively.

3. The dielectrophoresis detection device for detection and quantification of bacteria in water according to claim 1, wherein a liquid reservoir (204) is further arranged on the semiconducting microelectrode chip (200) close to the dielectrophoresis outlet.

4. The dielectrophoresis detection device for detection and quantification of bacteria in water according to claim 1, wherein the impedance inlet (203a) is arranged at one end of the semiconducting microelectrode chip (200) close to the dielectrophoresis outlet.

5. The dielectrophoresis detection device for detection and quantification of bacteria in water according to claim 1, wherein the dielectrophoretic actuator microelectrode part (201) is configured for detecting, manipulating, and classifying bacteria.

6. The dielectrophoresis detection device for detection and quantification of bacteria in water according to claim 5, wherein the impedance sensor microelectrode part (203) is configured for quantifying bacteria classified through the dielectrophoretic actuator microelectrode part (201).
